(19) United States
(12) Patent Application Publication
(10) Pub. No.: US 2021/0220239 A1
(43) Pub. Date: Jul. 22, 2021

(54) TABLETS CONTAINING POLYPHOSPHATES

(71) Applicant: Perfetti Van Melle S.P.A., Lainate (Milano) (IT)

(72) Inventors: Joaquin Lizano, KT Sittard (NL); Rene Hoeksema, KT Sittard (NL); Gladys Brouns, KT Sittard (NL); Andrea Sarrica, Lainate (Milano) (IT); Alessandro Bottini, Lainate (Milano) (IT); Guven Marangoz, KT Sittard (NL)

(73) Assignee: Perfetti Van Melle S.P.A., Lainate (Milano) (IT)

(21) Appl. No.: 17/055,192

(22) PCT Filed: May 16, 2019
(86) PCT No.: PCT/IB2019/054065

§ 371 Date: Nov. 13, 2020

(30) Foreign Application Priority Data
May 16, 2018 (IT) 1020180005426

(51) Int. Cl. A61K 8/24; A61K 8/02; A61K 8/34; A61Q 11/00

(52) U.S. Cl. CPC A61K 8/24; A61K 8/0216; A61K 8/345; A61K 2800/92; A61K 2800/412; A61K 2800/651; A61Q 11/00

(57) ABSTRACT

Disclosed is a confectionery product containing micronised polyphosphates. The products according to the invention have tooth-whitening properties and good palatability.

The invention relates to confectionery products, in particular tablets, containing at least one micronised polyphosphate.

### PRIOR ART

Compositions containing polyphosphates for the removal of tooth stains were described in U.S. Pat. No. 6,685,916, which suggests the use of one of the following polyphosphates together with other substances, including peroxide compounds and anionic surfactants: sodium tripolyphosphate, potassium tripolyphosphate, sodium pyrophosphate, sodium trimetaphosphate and sodium hexametaphosphate.

U.S. Pat. No. 6,685,916 deals with the problem of the unpleasant aftertaste typical of polyphosphates by proposing a solution based on the selection of specific doses of said polyphosphates.

Polyphosphates are also known to cause an accentuated residual roughness in the products containing them.

For example, U.S. Pat. No. 3,054,656, which refers in particular to the production of a pure sodium tripolyphosphate intended for use in the detergent industry, expressly mentions the question of roughness, which can be eliminated by processing sodium tripolyphosphate at very high temperatures under particular humidity conditions.

EP 1 227 789 describes confectionery products comprising polyphosphates, whose particle size is not stated, unlike that of the other ingredients, in particular silicates, used as abrasives in sizes ranging between 0.1 and 30 microns. Polyphosphates are only used in toothpastes, not in the tablets.

US 2003/003219 describes the use of polyphosphates with particle sizes ranging between 200 and 400 microns for the reduction of tooth stains.

US 2003/008062 describes chewable compositions for dental hygiene, in particular chewing gums, containing various ingredients including micronised polyphosphates inserted in a rough, crunchy matrix, comprising a large particulate able to give the desired rough, crunchy texture.

### DESCRIPTION OF THE INVENTION

It has now been discovered that the extreme roughness of tablets containing polyphosphates can be eliminated by using polyphosphates in micronised form.

Reducing the size of the polyphosphate considerably improves the sensation of roughness which would otherwise be perceived strongly and rather unpleasantly during consumption.

It has also been found that the problem of tablet roughness can be advantageously solved by directly pressing the micronised polyphosphates and at least one polyol, without granulation stages.

The invention therefore relates to a tablet comprising one or more polyphosphates in micronised form and one or more polyols. The tablets according to the invention, obtained by direct compression, have a smooth, not rough or crunchy texture, and dissolve in the oral cavity without chewing within a few minutes.

According to the invention, at least 90% by weight of the polyphosphates used to prepare the confectionery products, especially tablets, have a particle size of less than 150 μm.

The solution proposed by the invention differs from that proposed by U.S. Pat. No. 6,685,916, which in any event deals with the different technical problem of aftertaste, and from that proposed by U.S. Pat. No. 3,054,656, which solves the same technical problem but by a completely different method.

A micronised polyphosphate is preferably used wherein over 90% by weight of particles have a size of less than 150 μm, preferably over 90% by weight of the particles have a size of less than 106 μm, and more preferably over 80% by weight of the particles have a size of less than 53 μm.

Micronised polyphosphates are available on the market or can be obtained by subjecting commercial polyphosphates to conventional methods of micronisation, until the above-mentioned specifications are reached.

The tablet according to the invention does not contain sugar, but can contain any polyol, preferably sorbitol, xylitol and isomalt, either alone or mixed. Sorbitol is preferred, in percentages by weight ranging from 80 to 98%. It can also include flavourings, sweeteners and compression auxiliaries (lubricants and the like).

The polyphosphate contained in the tablet is selected from sodium tripolyphosphate, potassium tripolyphosphate, sodium pyrophosphate, sodium trimetaphosphate and sodium hexametaphosphate.

The tablet can also contain other active ingredients that do not interact with polyphosphates, such as vitamins. The tablet preferably does not contain mineral salts.

Hard tablets according to the following formulation were made by way of example:

| | Example 1 | Example 2 | Example 3 |
|---|---|---|---|
| | prior art | invention | invention |
| Ingredient | % | % | % |
| Sorbitol | 95.80 | 95.80 | 97.10 |
| Mint flavouring | 1.00 | 1.00 | 1.00 |
| Aspartame | 0.10 | 0.10 | 0.10 |
| Sucralose | 0.05 | 0.05 | 0.05 |
| Acesulfame K | 0.04 | 0.04 | 0.04 |
| Magnesium stearate | 1.00 | 1.00 | 1.00 |
| Sodium tripolyphosphate according | 2.00 | — | — |
| to prior art; 20% by weight of | | | |
| particles under 106 μm | | | |
| Sodium tripolyphosphate according | — | 2.00 | 0.70 |
| to the invention; 90% by weight of | | | |
| particles under 106 μm | | | |
| Brilliant blue | 0.01 | 0.01 | 0.01 |
| Total | 100.00 | 100.00 | 100.00 |

The tablets, weighing 700 mg each, were obtained by known methods with direct compression.

The products according to example 1 and example 2 were compared for roughness by a panel of thirteen expert tasters, according to the Quantitative Descriptive Analysis technique. The tasters blind-tasted one tablet for each example, 1 and 2, at separate times. They were required to suck the tablets for 3 minutes and score their roughness on a VAS scale from 0 (smooth) to 100 (rough) immediately, and after every minute's consumption. The scores are set out below:

| | Example 1 | Example 2 | |
|---|---|---|---|
| | prior art | invention | Example 3 |
| Minutes | % roughness | % roughness | invention |
| 0 | 51 | 41 | R ≤ 0.1 |
| 1 | 51 | 28 | R ≤ 0.1 |
| 2 | 49 | 25 | R ≤ 0.1 |
| 3 | 45 | 23 | R ≤ 0.1 |
| Overall opinion | Organoleptically | Organoleptically | |
| | unacceptable | acceptable | |

Due to the better palatability of the product, it is therefore possible to include an amount of polyphosphates, preferably ranging between 0.5% and 3% by weight of the total tablet weight, preferably between 0.6 and 2%, to make consumption of the tablet an effective method of reducing the formation of tooth stains, which is a further aspect of the invention.

The reduction in formation of tooth stains is demonstrated by an efficacy test involving sixty volunteers who tested the products according to examples 2 and 3 in a crossover procedure vs. no treatment (control). The test lasted for 12 weeks, at the start of which the volunteers had their teeth cleaned at a dentist's surgery to ensure an initial level of tooth stains amounting to zero. They then took 2 tablets four times a day (examples 2 and 3) o no tablet (control). The stains were measured on the front teeth after 12 weeks by the Modified Labelle Stain Index method, calculating the composite index of extension x intensity. It was thus possible to calculate the percentage reduction in stain formation compared with the control, the value of which is shown in the table below. The volunteers were questioned about the palatability of the tablets at the end of the period. The test results are set out below:

TABLE 1

| | Control | Example 2 | Example 3 |
|---|---|---|---|
| MLSI | 0.97 | 0.70 | 0.74 |
| % stain reduction | | 28% | 23% |

As shown in the table, the products according to the invention are therefore able to reduce the formation of tooth stains. None of the participants dropped out of the test, and all reported that the palatability of the tablets was acceptable.

1. Tablets comprising a micronised polyphosphate with at least 90% by weight of the particles having a diameter below 150 μm, and one or more polyols.

2. Tablets according to claim 1 obtained by direct compression.

3. Tablets according to claim 1 wherein the polyphosphate is present in percentages ranging from 0.5% to 3% by weight of the total product or tablet.

4. Tablets according to claim 1 wherein the polyphosphate is selected from sodium tripolyphosphate, potassium tripolyphosphate, sodium pyrophosphate, sodium trimetaphosphate and sodium hexametaphosphate.

5. Tablets according to claim 1 wherein over 90% by weight of the particles of the micronised polyphosphate have a diameter below 106 μm.

6. Tablets according to claim 1 wherein the polyol is sorbitol in percentages ranging from 80 to 98% by weight.

7. Method to reduce formation of tooth stain with the tablets of claim 1 in subjects in need thereof, said method comprising
orally administering said tablets and reducing formation of tooth stain is said subjects.

8. Tablets according to claim 5 wherein over 80% by weight of the particles have a diameter below 53 μm.
